(19) United States
(12) Patent Application Publication
(10) Pub. No.: US 2014/0114202 A1
(43) Pub. Date: Apr. 24, 2014

(54) BLOOD WITHDRAWAL CANNULA OF A PUMP REPLACING OR ASSISTING ACTIVITY OF THE HEART

(71) Applicants: Marc Hein, Aachen (DE); Greatex Nicholas, Aachen (DE); Roland Graefe, Aachen (DE); Ulrich Steinseifer, Hauset (BE)

(72) Inventors: Marc Hein, Aachen (DE); Greatex Nicholas, Aachen (DE); Roland Graefe, Aachen (DE); Ulrich Steinseifer, Hauset (BE)

(21) Appl. No.: 14/123,849

(22) PCT Filed: May 25, 2012
(86) PCT No.: PCT/EP2012/002245

§ 371 Date: Dec. 4, 2013

(30) Foreign Application Priority Data
Jun. 10, 2011 (DE) 102011106142.1

(51) Int. Cl. A61M 1/10; A61B 5/00; A61B 5/027; A61B 5/02; A61B 5/0215; A61B 5/029

(52) U.S. Cl. CPC A61M 1/1086; A61B 5/0215; A61B 5/029; A61B 5/027; A61B 5/02028; A61B 5/4836
USPC 600/486; 600/17

(57) ABSTRACT

The invention relates to a blood withdrawal cannula (4) for connecting a pump (2) assisting or replacing activity of the heart to the inner volume of a heart ventricle (1), in particular the left ventricle. At the end thereof that is located in the ventricle the cannula has a pressure sensor (7a, 7b) for measuring the ventricle pressure and/or ventricle pressure differences and at the same end of the cannula has a volume sensor (3a, 3b, 5, 6) for measuring the volume and/or volume changes of the ventricle (1) in at least a partial region of the ventricle. The invention further relates to a measuring device for monitoring the ventricle contractions and/or the function of a pump replacing or assisting activity of the heart. The measuring device can be/is connected to the pressure sensor (7a, 7b) and to the volume sensor of a blood withdrawal cannula according to any one of the preceding claims and is designed to detect pressure changes and volume changes of a ventricle (1) as the heart is beating.

The present invention relates to a blood-drawing cannula for connecting a pump that assists or replaces cardiac action to the chamber of a heart ventricle, in particular the left ventricle, which includes at the end of the cannula, placed inside the ventricle, a pressure sensor for measuring the ventricular pressure and/or ventricular pressure differences.

The invention further relates to a measuring apparatus for monitoring contractions of the ventricle and/or the function of a pump replacing or assisting cardiac action, as well as a method for adjusting the volume flow and/or the discharge pressure of a pump replacing or assisting cardiac action and transporting blood out of the heart ventricle and into the aorta of the heart by a blood-drawing cannula.

The publication WO 2008/140034 [US 2010/0160801] by Tokyo Medical and Dental University, for example, teaches the use of pumps in support of cardiac function, such as, for example, continuous-flow rotary pumps or any other type of pump. A pump of this kind transports blood out of the ventricle of the heart, for example out of the left ventricle, and to which end a blood-drawing cannula is placed inside the ventricle, particularly in the apex region of the heart, and through which the blood is transported from the ventricle into the pump. If connected to the left ventricle, the pump then transports the blood into the aorta.

The blood-drawing cannula of the above-mentioned publication includes a pressure sensor mounted at the end of the blood-drawing cannula that is inside the ventricle. It was found possible to evaluate ventricular contractions on the basis of the pressure and the motor output of the pump.

Newer pumps that assist and/or replace cardiac action use, for example continuous-flow rotary pumps. It is important therewith that the volume flow that is a function of the rotation speed of the pump and/or the discharge pressure be adjusted such that the heart function is optimally supported, in particular that the contraction of the ventricle is supported in order to thereby give the heart an opportunity to recover or heal or to help patients who are awaiting transplant.

The pumps that have been used to date are pumps with, for example substantially constant rotational speed and, therefore, constant volume flow/discharge pressure; these pumps are initially set up and adjusted by experienced hospital staff. A volume flow/discharge pressure of adequate size must be selected to ensure sufficient cardiac support, while these values cannot be set too high in an effort to avoid collapse of the ventricle. This adjustment can be determined, for example using additional sensors that are briefly introduced into the heart for this purpose and that cannot, however, dwell inside the heart for any extended periods of time. Moreover, sensors suffer from the problem that they drift over time, and an optimal volume flow of the pump can also vary depending on other orders of magnitude, for example as a function of the values indicating temperature, blood viscosity, etc. This is the reason why no device for assisting cardiac function that is suited for use as a long-term monitoring device has been available to date.

Therefore, it is the object of the present invention to provide an apparatus and a method for monitoring the volume flow/discharge pressure of the pump at any time in order to provide optimal cardiac support and/or monitor ventricular contractions.

This object is achieved, on the one hand, by providing that a blood-drawing cannula of the type as referred to in the introduction includes on the same end of the cannula that is inside the ventricle in addition to the pressure sensor a volume sensor for measuring the volume and/or volume changes of the ventricle at least in part of the ventricle.

The invention therefore provides as an essential aspect of the present invention for the possibility of monitoring and evaluating ventricular contractions at any time, meaning as long as a pump is used that supports cardiac action, particularly the work performed in the area of the left ventricle.

It is not necessary for the pressure sensor and/or the volume sensor to capture an absolute measured value; instead, according to the invention, it is sufficient to determine any changes related in the pressure and volume.

It was found that the work that is performed by the heart and/or the left ventricle of the heart can be represented by the area of a closed loop in the pressure-volume (PV) diagram of the ventricular contraction with each beat of the heart. This area is independent of the respective absolute pressure and volume values, whereby capturing any changes of these orders of magnitude is sufficient for measuring the cardiac work.

Furthermore, monitoring the total volume of the ventricle is also not necessary; instead, it is sufficient to measure just a part of the of the volume of the ventricle for changes, because there exists a relationship, particularly a linear relationship, between the volume change of the ventricle part and the total volume of the heart. This relationship can be taken into account in the calculation of the volume measuring values.

Therefore, using the sensors on the blood-drawing cannula, the invention offers the possibility of monitoring the ventricular contraction at any time, in particular in terms of the work performed by the ventricle, because the measured volume and pressure values can be taken at any time on the blood-drawing cannula, for example for the purpose of an external evaluation or evaluation by a measuring apparatus that is mounted on the blood-drawing cannula or the pump. This way, an attending physician can modify the operating speed of the pump and/or the volume flow/discharge pressure at any time, which can then be monitored by the measured values and/or the calculated work of the ventricle and/or heart or the change thereof over time.

It can be seen herein that, with an increase in the volume flow and/or pumping speed over an initial range, the cardiac work first increases, specifically over such a range in which the heart performs work to open the aortic valve against the pressure of the pump that discharges into the aorta, until the cardiac work decreases significantly, when a volume flow/discharge pressure is reached at which the heart valve remains closed.

Correspondingly, monitoring the cardiac work or any change thereof (as a function over time) on the basis of measured sensor values and ascertaining the point when the significant decrease of the cardiac work occurs, it is thus possible to select the volume flow/discharge pressure of the pump in such a way that a working point of the pump is achieved when the heart valve remains closed during the heartbeats.

This working point can be found as a function of the volume flow/discharge pressure, for example by staying below maximally measured cardiac work by a certain amount, as a percentage or absolute value, or by establishing and/or staying below a certain defined negative increase of the cardiac work (for example the difference quotient of cardiac work and pump speed/volume flow).

In one embodiment of the invention, the volume sensor can be provided as a pin that extends, facing away from the end of the cannula, particularly into the chamber of the ventricle and that includes on its surface two electrodes spaced longitudinally along the pin that can be used to generate, by applying a potential difference to the electrodes, a current flow through the blood of the ventricle, and the pin includes at least two sensing electrodes that are provided between these electrodes and used for measuring a voltage drop in the presence of a current flow between the sensing electrodes. The pressure sensor here can also be for example mounted on the pin or it can be mounted at the end of the cannula. Advantageously, in this configuration, it is possible to select the length of the pin and the arrangement of the electrodes thereon in such a manner that the electrodes that define the measuring range are provided centrally inside the ventricle, particularly for providing precise measurement results. Since heart sizes are not uniform and can differ from person to person, conceivably, the blood-drawing cannulas are provided in different sizes offering an assortment from which a suitable cannula can be selected depending on the size of the patient's heart.

In another embodiment, the cannula itself can be provided with two electrodes that are longitudinally spaced on the outer surface of the cannula at the end of the cannula that is placed inside the ventricle, such that by applying a potential difference, it is possible to generate a current flow through the blood of the ventricle, and at least two sensing electrodes are provided between these electrodes that are able to measure the voltage drop occurring between the sensing electrodes in the presence of a current flow. In this configuration, the pressure sensor is also provided at the end of the cannula.

Correspondingly, the voltage drop between at least two sensing electrodes can be used to determine the volume of the ventricle in a region surrounding the respectively addressed sensing electrodes. This is possible based on the fact that more blood (a larger volume) has a lower resistance, which is why the voltage drop in the blood volume surrounding the sensing electrodes is smaller in cases with more blood than in cases when a small blood volume surrounds the sensing electrodes.

As mentioned in the introduction, the distance between the electrodes to which the potential difference is applied for generating a current flow does not need to be as large as the total length of the ventricle, meaning a volume sensor does not need to extend through the total volume of the ventricle to be useful in calculating, using a plurality of sensing electrodes (sensing electrode pairs), the total volume of a ventricle.

Rather, the monitored ventricle length can be, in particular, smaller or equal to 50% of the total length of the ventricle between the cardiac apex and the aortic valve and still be sufficient. At least two sensing electrodes are provided in this area that serve for determining the partial blood volume surrounding the sensing electrodes, and this partial volume can then be used to extrapolate the actual volume on the basis of the linear relationships that were mentioned above.

According to the invention, a measuring apparatus can be provided that can be connected to the pressure and volume sensors of a blood-drawing cannula of the kind according to the invention as previously described, and that is designed to capture pressure and volume changes inside a ventricle, meaning able to detect the measured values of the above-mentioned sensors, in particular to measure and to store them. To this end, the measuring apparatus generates a voltage difference between the two outermost electrodes in order to thus generate a current flow; during the heartbeats, it measures the at least one voltage drop over the at least two sensing electrodes provided there between as well as, simultaneously, the pressure by the pressure sensor.

In an improvement, the measuring apparatus can be designed such that it is able to calculate, based on measured volume and pressure changes, the cardiac work. For example a measure of this kind can be made available to an attending physician, for example via a data readout from the measuring apparatus or a display, or the data are used for controlling or adjusting the pump directly. Furthermore, the measuring apparatus can be designed to output an adjustment signal for an adjustable volume flow/discharge pressure of a pump for replacing or assisting cardiac action that is based on measured volume and pressure changes, meaning, for example a signal for regulating the speed of the pump.

This is why the invention can be used for implementing a controlling or regulating process to capture the pressure and volume changes in the context of ventricular contractions by the use of pressure and volume sensors provided inside the ventricle and utilizing these changes to arrive at a measure for the current cardiac work, whereupon the volume flow/discharge pressure of the pump is adjusted and/or regulated as a function of this measurement.

To this end, the detected measured values can be supplied, for example to a filter and/or evaluation circuit in the measuring apparatus or an algorithm in an effort to determine the cardiac work. The adjustment and/or regulation of the volume flow and/or discharge pressure can be achieved, in particular, in that the aortic valve of the left ventricle is/remains closed during the ventricle contractions.

As mentioned previously, this object can be achieved by monitoring the measure of the cardiac work relative to the volume flow/discharge pressure and/or the rotational speed of the pump in order to establish a point after which the cardiac work drops significantly, while the volume flow increases. This is an indicator that after this point the aortic valve is closed.

It must be viewed as particularly advantageous that, according to the invention, no absolute measured pressure or volume values are needed for a determination of the measure of the cardiac work. This is why possible sensor drifts occurring over time cannot negatively affect the invention. Due to the fact that the pressure and volume sensors are each provided on a single element, namely the blood-drawing cannula of the heart pump, and since this element remains in place for the duration of the cardiac support treatment, the sensors do not create any additional stress for the patient, as would be the case if the sensors were placed through the aorta and into the heart.

This is the reason why the present invention is very well suited for monitoring, over long periods of time, the pump that replaces or assists cardiac action as well as the thus supported ventricle.

In addition to a determination of the cardiac work, it is also possible to determine any changes in the cardiac work, for example by the calculated derivation of the volume sensor signal (and/or the resulting calculated volume) over time. Minimum values of this change demonstrate an acute sensitivity with regard to the systole or contraction of the ventricle. Maximum values of this change are sensitive regarding the diastole or refilling of the ventricle. Correspondingly, these values can also be used for drawing conclusions as to the ventricular function. Not least of all, these values can be used as well for adjusting and/or regulating the speed of the pump, thereby changing volume flow and/or discharge pressure.

The measuring apparatus is generally designed in such a manner that it implements all the described method steps; in particular, it is possible, for example to provide a microprocessor in the measuring apparatus as well as software that executes the method steps.

Embodiments of the present invention will be described below:

FIG. 1 is a symbolic representation of the lower apex region of a heart with a blood-drawing cannula 4 inserted in the left ventricle 1. Using the blood-drawing cannula 4, a pump 2, for example a continuous-flow rotary pump, removes blood from the left cardiac ventricle 1 and feeds it to the aorta so that the pressure in the aorta increases. The pressure side of the pump 2 and the connection to the aorta are not shown here.

By the intrinsic cardiac contraction of the ventricle, the heart transports the blood, countering this pressure, through the aortic valve until the pump has generated a pressure in the aorta against which the heart can no longer pump. After this pressure is achieved, stress is removed from the heart, and the pumping function is handled by the pump.

A pressure sensor 7b and a volume sensor 3b are provided on the blood-drawing cannula 4. The volume sensor 3b is here configured as a pin 3b that extends at least partially from the cannula into the ventricle 1 and carries on its outer surface a plurality of electrodes 5 and 6 that are spaced axially apart. The pressure sensor 7b is here mounted at the extreme outer end of the pin 3b.

A potential difference or voltage can be applied to the electrodes 5 that are most widely spaced from each other in order to thereby generate a current flow through the surrounding blood volume.

The voltage drop, which is measurable between two respective electrodes 6, two of which are provided at least between the electrodes 5, decreases or increases depending on the surrounding blood volume. The pressure drop of one pair or a plurality of pairs of electrodes 6 thus constitutes a measure of the blood volume that is around the electrodes 6.

On the basis of a preferably linear relationship, it is possible, as provided in an improvement of the present invention, to recalculate the total volume. This relationship can be established, for example in a preparatory step using a sensor that measures the entire cardiac volume. It is also possible to omit such a step.

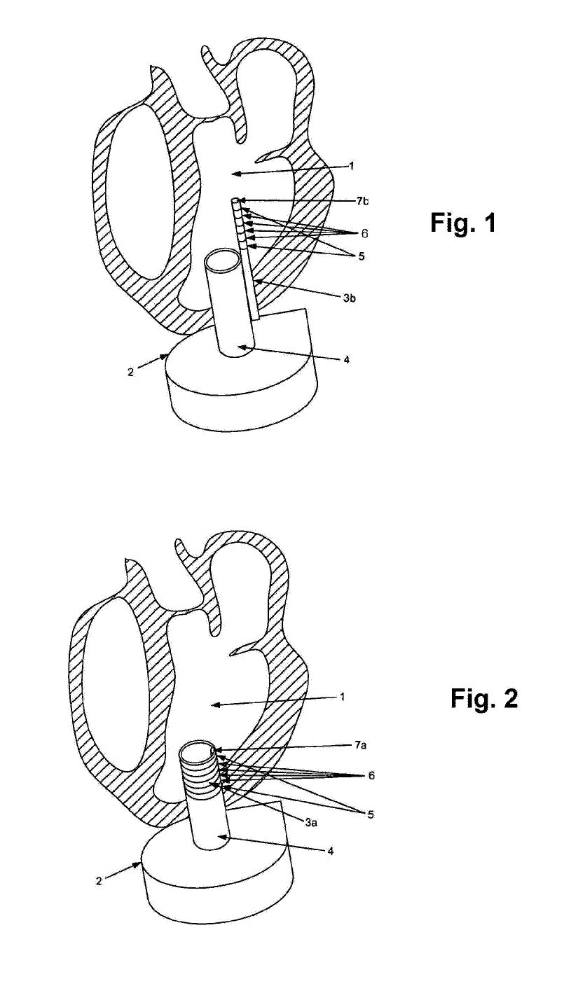

FIG. 2 shows an alternate solution where the previously mentioned electrodes 5 and 6 are not mounted on a pin that is fastened to the end of the cannula; instead, the same electrodes 5, 6 are provided directly on the outer surface of the cannula 4 that has been inserted deeply into the ventricle 1. The measurement of the (partial) volume of the ventricle is handled in the same manner as before. In this embodiment, the pressure sensor 7a is mounted internally at the outer end of the cannula 4.

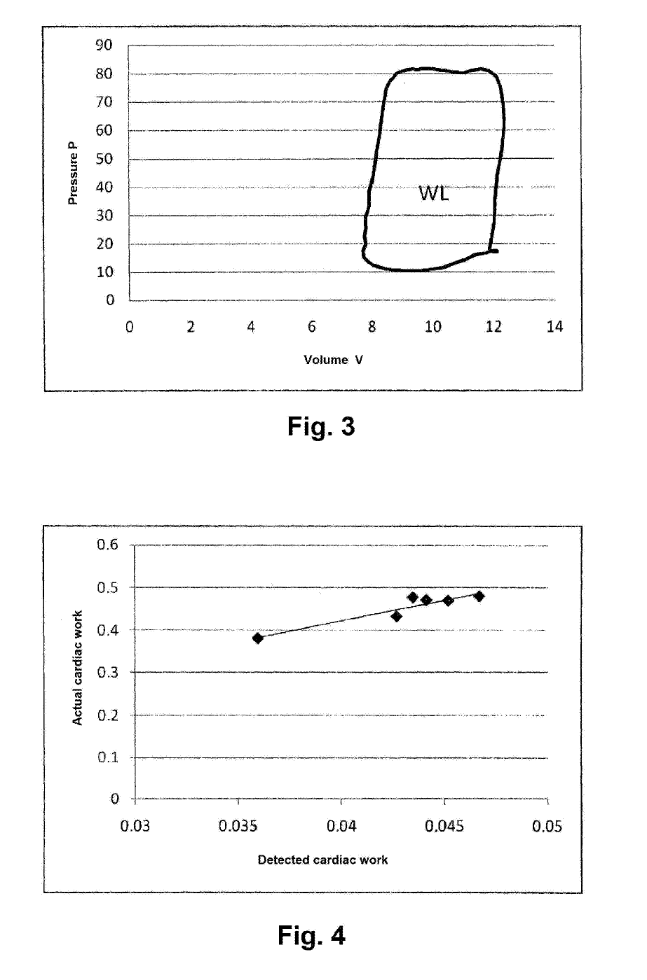

FIG. 3 is a PV diagram for a heartbeat of the heart with regard to the measured values of pressure P and volume V that were detected in the ventricle. The cardiac work WL that is performed by the heart can be derived from the area of the closed loop and can be mathematically established, for example by integration. This area is visibly independent of the absolute pressure and volume values, which is why the invention does not require the use of absolutely calibrated sensors. As previously mentioned, it is possible to draw conclusions as to the total volume by measuring a partial volume range of the ventricle.

Due to the fact that a volume-sensor of the above-mentioned kind does not allow for establishing absolute volume values, the determined measure of the cardiac work is also not an absolute order of magnitude. However, FIG. 4 demonstrates that there exists a linear relationship between the measured cardiac work and the actual cardiac work; this is the reason why the cardiac work that was calculated based on the method according to the invention using the apparatus according to the invention can be used as a basis for controlling the pump with or without feedback.

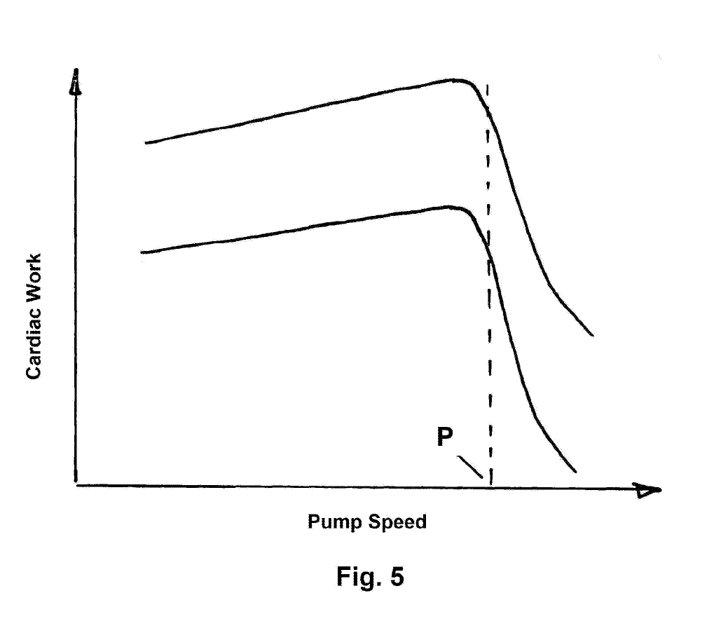

FIG. 5 shows the relationship between the cardiac work (Y-axis) and the speed of the pump (X-axis) that influences the volume flow and/or discharge pressure of the pump. Curve A indicates the relationship based on cardiac work that was detected when the total ventricle volume was established; curve B shows this relationship, using the above volume sensor, based only on a partial volume.

This shows that, with increasing rotational speed, both curves A, B climb slightly initially, then drop off steeply upon reaching the same speed. As of this point in time, which is here marked as point P, the aortic valve is closed and the cardiac work decreases. As of this point, effective heart assistance applies. Therefore, preferably, the (partial) volume change and pressure change of the cardiac work is measured according to curve B, selecting a pump speed after which the aortic valve is closed.

Due to the fact that the cardiac work transitions from a left-side plateau into a steep drop, this point P can be detected, for example by comparing the cardiac work with a stored reference value. If the negative angle is smaller or the amount of the angle is greater than the reference value, this working point of the pump has been reached and/or exceeded. A sign change in the angle can be used as well for testing purposes. In the area of the plateau, the angle is slightly positive, whereas in the vicinity of point P, the angle becomes markedly negative.

Various possibilities are presently conceivable for determining point P mathematically on the basis of the measured values. In addition, depending on the applicable medical indication, it is possible to exceed point P more or less in the direction toward higher speeds. The amount can be defined, for example by the physician and stored in the measuring apparatus as a rule parameter.

1. A blood-drawing cannula for connecting a pump assisting or replacing cardiac action to the chamber of the left ventricle, with, at the end of the cannula inside the ventricle, a pressure sensor for measuring the ventricle pressure and/or ventricle pressure differences, wherein the cannula has a volume sensor at the same cannula end for measuring the volume and/or volume changes of the ventricle in at least a part of the ventricle.

2. The blood-drawing cannula according to claim 1, wherein the volume sensor is configured as a pin that extends, facing away from the cannula end, particularly into the chamber of the ventricle, and includes two spaced electrodes that are mounted on the surface thereof by means of which, due to the application of a potential difference, a current flow can be generated through the blood of the ventricle, and the pin includes at least two sensing electrodes that are provided between these electrodes by which a drop in voltage, occurring in the presence of a current flow between the sensing electrodes, can be measured.

3. The blood-drawing cannula according to claim 1, wherein the cannula itself includes, at the end thereof located inside the ventricle, on the outer surface thereof, two electrodes spaced longitudinally along the cannula by which, with application of a potential difference, a current flow can be generated through the blood of the ventricle, and at least two sensing electrodes are provided between them by means of which a voltage drop can be measured between the sensing electrodes that occurs in the presence of a current flow.

4. A measuring apparatus for monitoring the ventricular contractions and/or the function of a pump that replaces or assists cardiac action, wherein the pump is/can be connected to the pressure sensor and volume sensor of a blood-drawing cannula according to any one of the preceding claims and is designed to detect pressure and volume changes of a ventricle as the heart is beating.

5. The measuring apparatus according to claim 4, wherein the device is designed to calculate the cardiac work and/or the change of cardiac work on the basis of the measured volume changes and pressure changes.

6. The measuring apparatus according to claim 4, wherein the measuring apparatus is designed to output, based on the measured volume and pressures changes, an adjustment signal for a volume flow and/or discharge pressure of a pump that replaces or assists cardiac action.

7. A method for adjusting a volume flow and/or discharge pressure of a pump that replaces or assists cardiac action and that transports blood out of the ventricle of the heart and into the aorta through a blood-drawing cannula, wherein the pressure and volume changes during the ventricular contraction are detected by pressure sensors and volume sensors mounted on a blood-drawing cannula inside the ventricle, and that these changes are used for determining a measure of the current cardiac work and/or the change of the cardiac work, and that the volume flow and/or discharge pressure of the pump is/are adjusted and/or regulated as a function of this measure.

8. The method according to claim 7, wherein the adjustment and/or regulation is achieved in that the aortic valve of the left ventricle is/remains closed during the ventricular contractions.
